(19) United States
(12) Patent Application Publication
(10) Pub. No.: US 2014/0236035 A1
(43) Pub. Date: Aug. 21, 2014

(54) METHOD AND SYSTEM FOR AUTOMATED VENTILATOR MONITORING TO ENSURE LUNG-PROTECTIVE VENTILATION

(71) Applicant: COVIDIEN LP, Mansfield, MA (US)

(72) Inventors: Gary S. Milne, Louisville, CO (US); Robert T. Boyer, Longmont, CO (US)

(73) Assignee: COVIDIEN LP, Mansfield, MA (US)

(21) Appl. No.: 13/767,948

(22) Filed: Feb. 15, 2013

(51) Int. Cl. A61B 5/08; A61M 16/00

(52) U.S. Cl. CPC A61B 5/08; A61M 16/0057
USPC 600/529

(57) ABSTRACT

A method and a system for acquiring patient parameters and automatically monitoring a patient's lung injury risk include a processor and processing instructions executable by the processor to periodically and automatically determine an arterial oxygenation parameter of the patient based on first data acquired from a monitoring device coupled to the patient. The method and system determine that the mechanical ventilator is set to ventilate the patient; evaluate a lung injury risk monitoring protocol associated with the patient, the lung injury risk monitoring protocol including the arterial oxygenation parameter; and provide a lung injury risk indication when the lung injury risk monitoring protocol is satisfied.

### TECHNICAL FIELD

The disclosure relates generally to a method and a system for monitoring a patient's breathing parameters. More particularly, the disclosure relates to a method and a system for automatically monitoring a patient's lung injury risk.

### BACKGROUND

Mechanical ventilation is a common life-saving technique in which a ventilator provides pressurized respiratory gases to patients to assist their breathing. Respiratory gases may include fresh air, scrubbed air, and anesthetics, for example. Most patients suffering from acute lung injury (ALI) or acute respiratory distress syndrome (ARDS) require mechanical ventilation. A risk associated with mechanical ventilation is ventilator induced lung injury (VILI).

It has been shown that VILI is more prevalent when a tidal volume (Vₜ) of 12-15 mL/kg of actual body weight is used for patients with ALI/ARDS. Subsequent studies have shown improved outcomes when lung-protective ventilation is used, which generally involves application of a reduced tidal volume. However, use of lung-protective ventilation does not eliminate the VILI risk, which is often unrecognized.

### SUMMARY

An automated lung-protective monitoring method and system are provided herein. In one embodiment according to the disclosure, a system to automatically monitor lung-protective ventilation of a patient comprises a processor; a computer readable storage medium; and processing instructions embedded in the computer readable storage medium and executable by the processor. The processing instructions are executable by the processor to periodically and automatically determine an arterial oxygenation parameter of the patient based on first data acquired from a monitoring device coupled to the patient; determine that the mechanical ventilator is set to ventilate the patient; evaluate a lung injury risk monitoring protocol associated with the patient, the lung injury risk monitoring protocol including the arterial oxygenation parameter; and provide a lung injury risk indication when the lung injury risk monitoring protocol is satisfied.

The above-mentioned and other disclosed features which characterize the embodiments of the system and method described herein advantageously leverages continuous ventilation monitoring and clinical knowledge of ventilation modes to recognize when a patient is exposed to lung injury risk. Continuous monitoring speeds the detection of risk conditions and reduces the time that a patient is exposed to damaging conditions. Continuous monitoring, as described, also reduces the noise inherent when sampling only a few data points. A further advantage is the provision of configurable alarms, based on the continuous monitoring, which can potentially save a patient's life by alerting a healthcare provider to the potential injury before it occurs. The configurable time periods enable healthcare providers to balance risk awareness against nuisance alarms. Mechanical ventilator settings may be monitored to reset timing periods when important changes are made, which is important because often VILI risk is highest immediately following ventilator setting changes or patient movement. A further advantage is the standardization of the process of, and protocols for, monitoring a patient's exposure to lung injury risk, and the potential refinement of such protocols.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some or none of the enumerated advantages.

### BRIEF DESCRIPTION OF THE DRAWINGS

The above-mentioned and other disclosed features, and the manner of attaining them, will become more apparent and will be better understood by reference to the following description of disclosed embodiments taken in conjunction with the accompanying drawings, wherein:

FIG. 1 is a block diagram of an embodiment of a system according to the disclosure for automatically monitoring a patient's lung injury risk;

FIGS. 2-4 are diagrammatic representations of embodiments of a graphical user interface according to the disclosure for automatically monitoring a patient's lung injury risk;

FIGS. 5 and 6 are diagrammatic representation of embodiments of a graphical user interface according to the disclosure for editing lung injury risk monitoring protocols;

FIG. 7 is a flowchart of an embodiment of a method according to the disclosure for automatically monitoring a patient's lung injury risk;

FIG. 8 is a block diagram of an embodiment of a software product according to the disclosure; and

FIG. 9 is a block diagram of another embodiment of a system according to the disclosure for automatically monitoring a patient's lung injury risk.

Corresponding reference characters indicate corresponding parts throughout the several views. Although the drawings represent embodiments of various features and components according to the present disclosure, the drawings are not necessarily to scale and certain features may be exaggerated in order to better illustrate and explain the present invention. The exemplification set out herein illustrates embodiments of the disclosure, and such exemplifications are not to be construed as limiting the scope of the invention in any manner.

### DETAILED DESCRIPTION

For the purposes of promoting an understanding of the principles of the invention, reference will now be made to the embodiments illustrated in the drawings, which are described below. The embodiments disclosed below are not intended to be exhaustive or limit the invention to the precise form disclosed in the following detailed description. Rather, the embodiments are chosen and described so that others skilled in the art may utilize their teachings. It will be understood that no limitation of the scope of the disclosure is thereby intended. The invention includes any alterations and further modifications in the illustrated devices and described methods and further applications of the principles of the invention which would normally occur to one skilled in the art to which the invention relates.

The transitional term “comprising”, which is synonymous with “including,” or “containing,” is inclusive or open-ended and does not exclude additional, unspecified elements or method steps. By contrast, the transitional term “consisting” is a closed term which does not permit addition of unspecified terms.

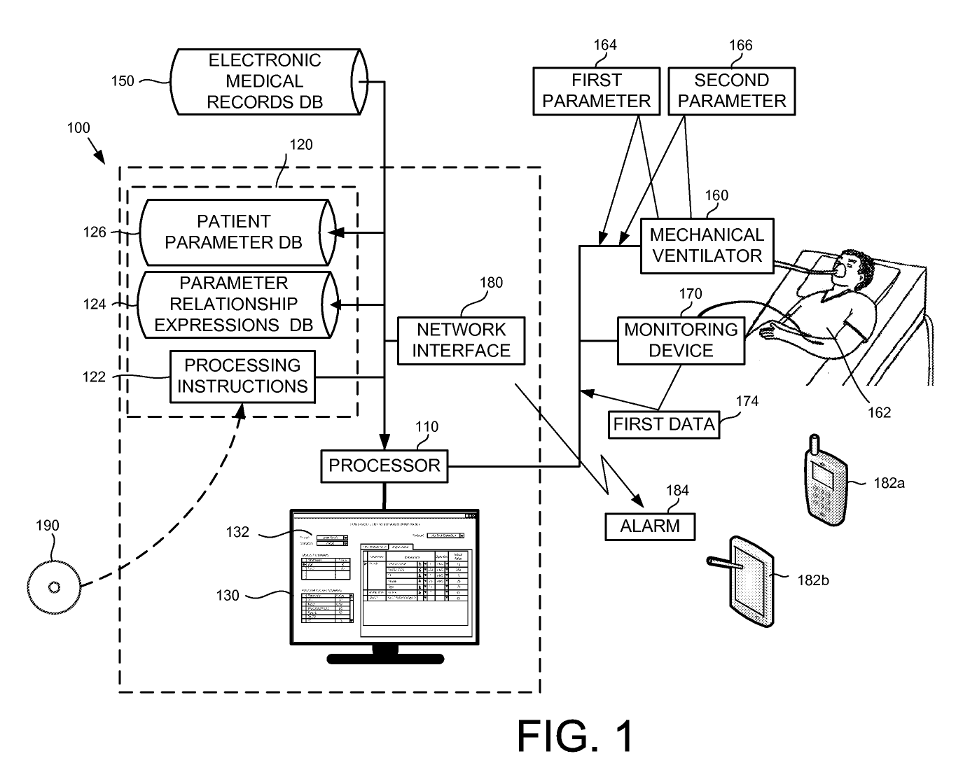

Referring to FIG. 1, a diagram is presented of an embodiment of a system according to the disclosure, denoted by numeral 100, for automatically monitoring a patient's lung injury risk. System 100 comprises a processor 110 coupled to a computer readable storage medium 120 having stored therein processing instructions 122 executable by processor 110 to monitor the patient's lung injury risk. System 100 also comprises a patient parameter relationship expressions database 124 including a lung injury risk expression. System 100 is communicatively coupled to a mechanical ventilator 160 operable to ventilate a patient 162 and to a monitoring device 170 operable to acquire, periodically and automatically, a first data 174 from patient 162. System 100 may also operable to obtain a first parameter 164 and second parameter 166 from mechanical ventilator 160. System 100 may also comprise a patient parameter database 126 operable to store patient parameters obtained from mechanical ventilator 160 and monitoring device 124. First data 174 may comprise an arterial oxygenation parameter or data operable to determine the arterial oxygenation parameter. In one example, monitoring device 170 comprises a pulse oximeter and first data 174 comprises an oxygen saturation parameter and/or information operable to determine the oxygen saturation parameter. First parameter 164 may comprise information operable to determine that patient 162 is being ventilated. Second parameter 166 may comprise information operable to determine ventilation modes or conditions. Exemplary modes or conditions include volume or pressure mode, tidal volume, inspired oxygen fraction, and any other information provided by mechanical ventilator 160.

In operation, system 100 communicates with mechanical ventilator 160 to determine that mechanical ventilator 160 is set to ventilate patient 162, to evaluate at least a lung injury risk expression, and to determine if the lung injury risk expression indicates a lung injury risk exposure. If exposure is indicated, system 100 tracks an exposure parameter. Exemplary exposure parameters include exposure time and exposure ratio. System 100 then compares the exposure parameter to an exposure threshold and provides a lung injury risk indication when the exposure parameter equals or exceeds the threshold. The lung injury risk expressions, including an expression having the exposure parameter, may be referred to as a lung injury risk monitoring protocol. In one example, in which the exposure threshold comprises a predetermined amount of time, e.g. 7 minutes, system 100 provides a lung injury risk indication when the exposure time equals or exceeds 7 minutes. The exposure time may be accumulated over a moving window of time, such that exposure time reflects exposure over the length of the moving window. For example, the moving window may be defined as two hours, and the lung injury risk indication may be given if the cumulative exposure time during the last two hours exceeds 7 minutes. In another example, the exposure parameter is an exposure ratio expressed as the percentage of the exposure time over a predetermined time. For example, a 15% exposure ratio, with a 60 minute period, is achieved when a cumulative exposure time equals or exceeds 9 minutes during the last 60 minute period. System 100 may provide a lung injury risk indication when the exposure ratio exceeds the exposure ratio threshold, e.g. 15%. An exposure ratio enables system 100 to provide a lung injury risk indication even though the lung injury risk exposure is not continuous.

The lung injury risk indication may comprise a message or an alarm transmitted to a processing device accessible to a healthcare provider. Exemplary processing devices include network clients, mobile devices and any other processing device adapted to receive messages. The healthcare provider may suspend the alarm for a predetermined amount of time, e.g. 12 hours. The suspension may be revoked by system 100 upon the occurrence of an event, which may be referred to as the alarm suspension revocation event. Exemplary alarm suspension revocation events include a change in critical mechanical ventilator settings (e.g. volume, pressure, and other inspiratory settings) and a change in the location of the patient. Changes in healthcare provider duties and roles may also comprise an alarm suspension revocation event. For example, an alarm suspended by one healthcare provider may be reinstated when the healthcare provider goes off duty so that the healthcare provider coming on duty will be alerted if the alert condition remains in effect.

System 100 may present a plurality of views with a user interface. A view may include a drop-down list of patients that may be selected to enable a healthcare provider to switch the presentation of information for different patients, a drop-down list of protocols to enable a healthcare provider to quickly switch from one protocol to another, data entry fields to enter patient parameters, and one or more indicators to provide constructive notice of the lung injury risk exposure and the time of exposure. An exemplary graphical user interface (GUI) 132 is shown. GUI 132 is operable to define and monitor lung injury risk monitoring protocols, select and modify protocols, and associate the protocols with patients. GUI 132 may be presented on a computer display 130 coupled to processor 110. As shown, computer display 130 is coupled to processor 110 by a wired connection. GUI 132 may also be presented by mechanical ventilator 160 or a processing device in any known or later developed manner. System 100 may provide a lung injury risk indication with GUI 132. For example, GUI 132 may display the exposure time in one color and change the color when the exposure threshold is satisfied. System 100 may also provide an audible or visual lung injury risk indication by communicating a message to a mobile device, to mechanical ventilator 160 or to a facility annunciation system. The annunciation system may be based on message routing rules. The annunciation system may include role-based alarm levels and designated alarm recipients to manage the alarms.

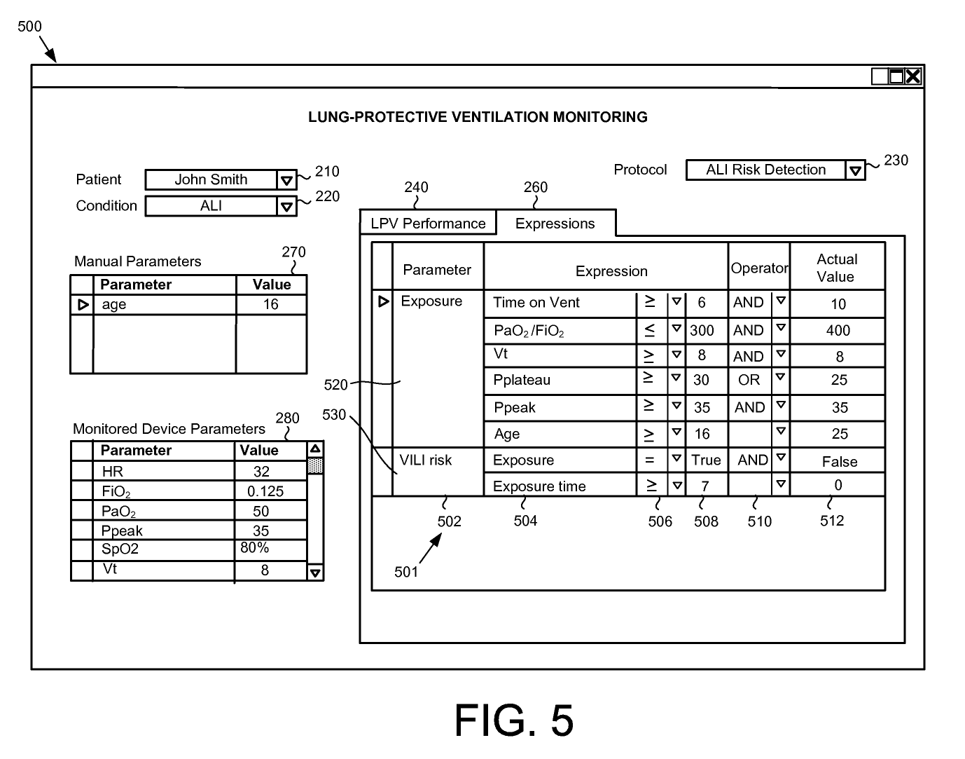

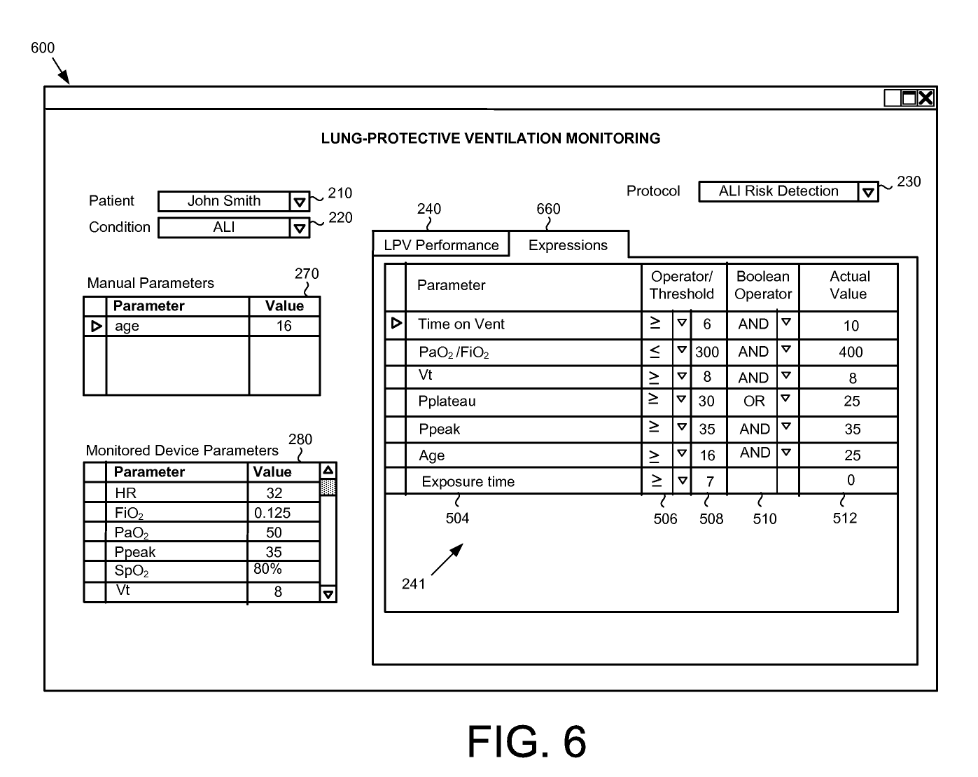

A lung injury risk monitoring protocol may be associated with a patient to display data corresponding to the protocol. A standard lung injury risk monitoring protocol may be automatically associated with a patient coupled to a ventilator. A nurse or other healthcare provider may associate the patient with the ventilator. Further, a lung injury risk monitoring protocol may be associated with a patient by selecting the patient, selecting the protocol, and then saving or storing the association in a patient configuration file. A lung injury risk monitoring protocol may comprise a patient parameter relationship expression or several expressions associated by Boolean logic operators, e.g. and/or, as show on FIGS. 5 and 6. GUI 132 may also provide start and stop monitoring controls operable by a healthcare provider to initiate monitoring and ensure lung-protective ventilation. In one example, monitoring begins automatically upon association of a patient with a protocol. In another example, monitoring begins automatically upon determination by system 100 that all the parameter acquisition apparatus required to evaluate a protocol are providing the required signals. In a further example, upon determination based on second parameter 166 of a change in ventilation parameters, monitoring begins based on alternate expressions. By alternate expressions it is meant that alternate expressions or thresholds are applied based on the change. In one variation, two protocols are associated with a patient, and system 100 includes instructions to switch between them. GUI 132 may include an option to associate the alternate protocol with the patient and parameters to determine when to switch to the alternate protocol. In another variation, a protocol includes alternate logic branches and logic to select an alternate branch based on the change.

System 100 may also comprise a network interface 180. Monitoring device 170 and mechanical ventilator 160 are shown coupled to system 100 by a wired network. Monitoring device 170 and mechanical ventilator 160 may include wireless transceivers and be coupled to system 100 by a wireless network via network interface 180. Network interface 180 may also communicate with a facility annunciation system and with processing devices including mobile devices, and transmit messages and/or an alarm 184 thereto. Exemplary mobile devices include a smart phone 182a, an electronic tablet 182b, and any other mobile device with wireless communication capability.

System 100 may receive patient parameters from a plurality of data sources including a medical records database 150, other patient monitoring devices, and user interfaces. Medical records database 150 may contain patient information such as age and medical condition, and patient parameters based on, for example, laboratory analysis of the patient's fluids. Patient monitoring devices may automatically record patient parameters such as heart rate and blood pressure, for example. Patient monitoring devices may be included in, or be part of, mechanical ventilator 160. User interfaces enable entry of patient parameters based on, for example, observation of the patient's state or performance of patient maneuvers. System 100 may periodically acquire parameter values from the various sources and store them in patient parameter database 126.

Each patient parameter relationship expression describes a relationship between at least one patient parameter and a threshold. The relationship comprises an operator and may comprise a single parameter (e.g. A≧c, where A is the parameter and “c” is a threshold), more than one parameter (e.g. A+B≧c, where A and B are parameters and “c” is a threshold), a function (e.g. f(A)≧c, where f(A) is any function of parameter A and “c” a threshold), and any combination of the foregoing. Of course, the operator may represent an inequality as well as an equality relationship. The system may comprise functions which users may include in the expressions. For example, a function may be provided to convert a parameter, and the conversion function may then be used in a patient parameter relationship expression. The expressions may also include ranges, e.g. {(K1<A<K2) AND (A+2B)≦c}, where A and B are parameters, K1 and K2 are constants and “c” is a threshold; therefore, the expression may be satisfied by a specified range of values of A. Ranges may also be defined in a function comprising Boolean algebra, where the threshold is a logical outcome, e.g. yes/no. Expressions may also include functions representing time durations, ratios and/or compliance requirements. For example, an expression may include a function requiring that a parameter exceed a threshold for a predetermined time or fall within a range for a predetermined time. The expressions may be defined and transformed to match clinical thought and the styles used in medical literature and references. For example, function variables may scale and normalize parameters. Also, the function variables may consolidate parameters from different sources to simplify configuration of the patient parameter relationship expressions and protocols.

The status of a patient parameter relationship expression is determined by comparing the actual parameter value (or result of the function) to the threshold based on the operator. Thus, the patient parameter relationship expression may be satisfied if the condition specified by the operator is satisfied or unsatisfied if the condition is not satisfied. In the event that the patient parameter is not yet available, a patient parameter relationship expression status may indicate that the evaluation of the expression is incomplete. In the present context, the terms satisfied, unsatisfied and incomplete are only exemplary. Any other suitable terms may be used to denote the status of an expression or protocol. For example, Boolean logic terms such as true/false or yes/no may be used instead of satisfied and unsatisfied.

System 100 may also comprise a program or processing instructions or sequences of instructions configured to cause processor 110 to determine and indicate the status of patient parameter relationship expressions and protocols based on the patient parameters. Exemplary status indicators may comprise icons of different colors or shapes, flashing icons, text messages, audible indications, and any other means for providing constructive notice to a healthcare provider concerning the satisfaction of a protocol associated with a patient. In one example, expression status indicators are presented to indicate which expressions have been satisfied and which have not been satisfied. Satisfaction of a protocol is indicative of a lung injury risk higher than normal.

Also shown on FIG. 1 is a computer program product 190 comprising a computer readable storage medium having computer readable processing instructions 122 embedded therein. Processing instructions 122 may be transferred to computer readable storage medium 120. In one example, computer program product 190 is a disk insertable into a disk drive of system 100 to be read by system 100. Computer program product 190 may also include parameter relationship expressions database 124 and patient parameter database 126. In one variation, computer program product 190 comprises instructions configured to periodically and automatically determine an arterial oxygenation parameter of the patient based on first data acquired from a monitoring device coupled to the patient; determine that the mechanical ventilator is set to ventilate the patient; evaluate a lung injury risk monitoring protocol associated with the patient, the lung injury risk monitoring protocol including the arterial oxygenation parameter; and provide a lung injury risk indication when the lung injury risk monitoring protocol is satisfied. In one example, the evaluation comprises determining that the mechanical ventilator is set to ventilate the patient. In one variation, the processing instructions are further configured to reset tracking of the exposure parameter if a ventilating parameter of the mechanical ventilator is changed. Exemplary ventilation parameter changes include switching from volume to pressure control or vice versa, and changing the tidal volume. In a further variation, the processing instructions are further configured to evaluate an alternate expression if a ventilating parameter of the mechanical ventilator is changed.

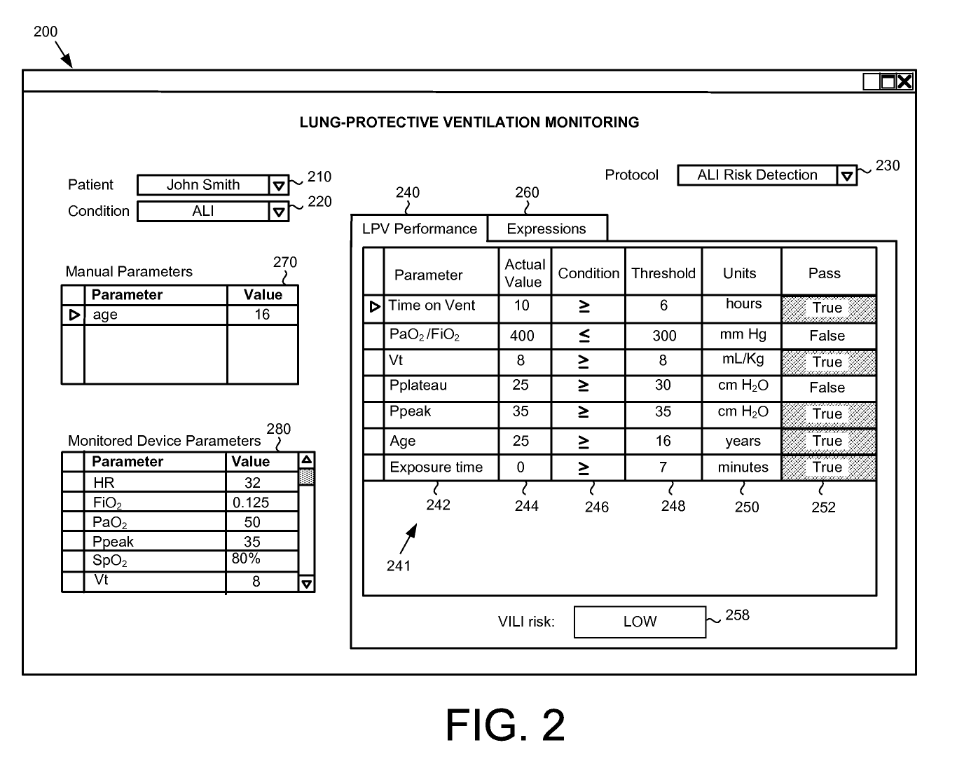

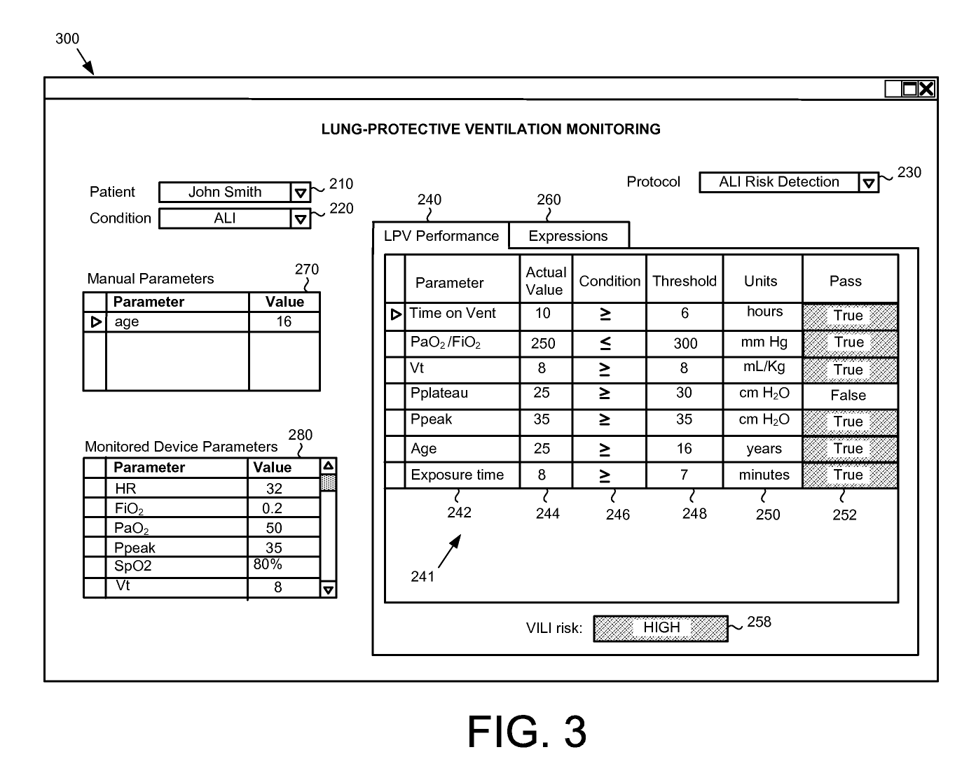

FIGS. 2-4 show views 200, 300 and 400, which are diagrammatic representations of embodiments of a graphical user interface according to the disclosure for automatically monitoring a patient's lung injury risk according to a lung injury risk monitoring protocol 241. Views 200 and 300 are similar except for the status of one parameter of a patient parameter relationship expression (PaO₂/FiO₂) and the corresponding status of a VILI risk indicator 258. In view 200, the expression is not satisfied so VILI risk indicator 258 indicates a low VILI risk. In view 300, the PaO₂/FiO₂ expression is satisfied, and VILI risk indicator 258 indicates a high VILI risk. The high risk is indicated even though the expression of parameter Pplateau is not satisfied because, as shown on FIGS. 5 and 6, Pplateau is subject to a Boolean OR condition. Views 200 and 300 illustrate an embodiment in which the VILI risk exposure time and an exposure time threshold are shown in the patient parameter relationship expression, while in view 400 the VILI risk exposure time is shown separately in box 410 and a “time to high risk” value, which is a count-down value equal to the difference between the exposure time and the exposure time threshold, is shown in a box 420. The exposure time and threshold may be included in one patient parameter relationship expression, as shown in FIGS. 2 and 3. The elements of views 200, 300 and 400 will now be described.

Views 200, 300 and 400 are configured to facilitate selection of protocols, association of a selected protocol with a patient, and monitoring of parameters and the patient parameter relationship expressions. Views 200, 300 and 400 include a patient selection box 210, a condition selection box 220, a protocol selection box 230, patient parameter panels 270 and 280, tabs 240 and 260 and VILI risk indicator 258. Selection of tab 240 enables a user to view the status of a selected protocol and the VILI risk indication corresponding to the status of the protocol. Selection of tab 260 enables a user to configure a protocol. In views 200, 300 and 400, tab 240 has been selected.

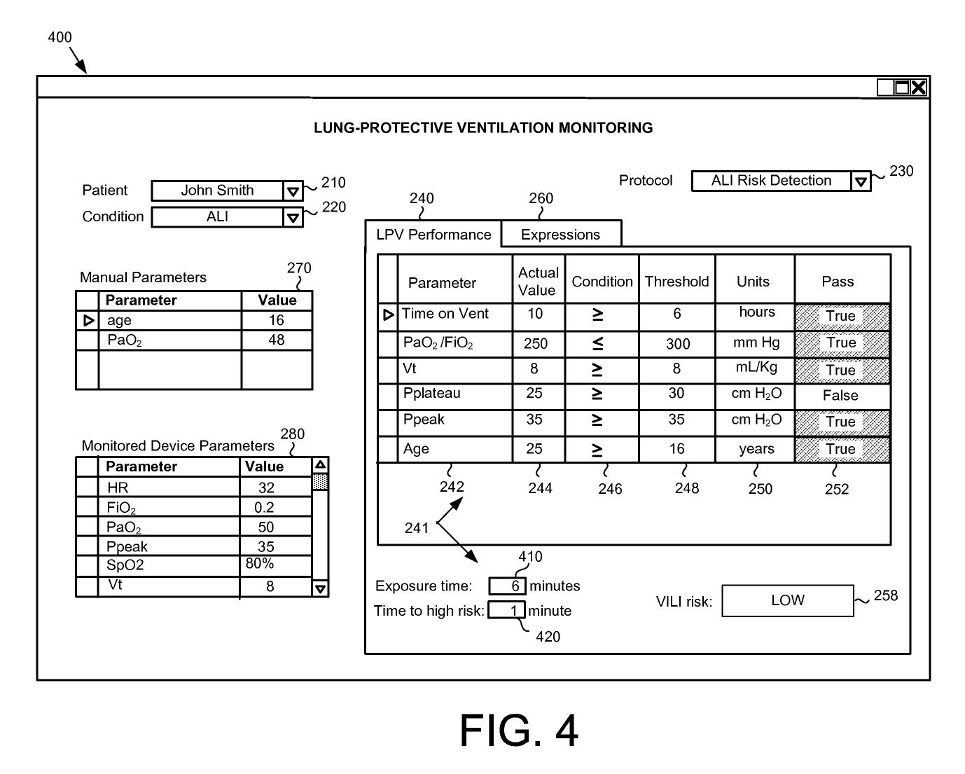

Patient selection box 210 and protocol selection box 230 enable users to select patients and protocols from drop-down lists. Condition selection box 220 enables a user to select a condition. System 100 may utilize the selected condition to filter available protocols and only make available via protocol selection box 230 those protocols matching the condition. The selections may be stored in patient specific configuration files in patient parameter relationship expressions database 124 or on any other suitable storage location. Patient parameter panels 270 and 280 display patient parameters corresponding to, respectively, manually entered parameters and parameters acquired with monitored devices. Other parameter panels may be provided to enter maneuvers, patient states or any other suitable parameter category. As shown in FIG. 4, the same parameter (e.g. PaPO₂) may be shown in multiple parameter panels if they are acquired by different means.

Tab 240 enables a user to monitor lung-protection ventilation. In views 200 and 300, tab 240 includes a table having a plurality of expressions of a selected lung injury risk protocol 241, the table having a plurality of columns 242, 244, 246, 248, 250 and 252 corresponding to, respectively, a parameter, actual values of the parameter, the condition operator of the expression, the threshold of the expression, the units of the parameter and the status of the expression. Each expression of protocol 241 is presented in a row. Expression status indicators may include text (true/false) and a color indicative of satisfaction of the expression, e.g. red, or non-satisfaction, e.g. green. Referring to FIG. 4, in view 400 the table shows an exposure portion of protocol 241 and, as discussed above, the exposure parameter is shown separately in box 410.

A protocol named ALI risk detection is shown, comprising a plurality of parameters (and corresponding expressions) including time on the ventilator, partial pressure of arterial oxygen (PaO₂), a ratio of partial pressure of arterial oxygen (PaO₂) to an inspired oxygen fraction (FiO₂), PaO₂/FiO₂, tidal volume (Vₜ), plateau pressure (Pplateau), peak airway pressure (Ppeak), the minimum age of the patient, and an exposure time. The FiO₂ and Vₜ parameters may be obtained from the mechanical ventilator. The exposure time is an exemplary exposure tracking parameter that begins to track time when the other expressions of the protocol indicate a lung injury risk exposure. If the other expressions include a time-based parameter, a lung injury risk indication may be provided when the protocol is satisfied, even if the protocol does not explicitly include an exposure expression. The cells in column 252 that are filled indicate a color difference from the color of the unfilled cells. The fill color may be red to indicate satisfaction of the particular expression. In one example, the protocol comprises: Time on ventilator≧6 hours, and PaO₂/FiO₂≦300 mm Hg, and Vₜ≧8 mL/kg of ideal body weight, and Pplateau≧30 cm HO₂ or Ppeak≧35 cm H₂O, and Age≧16 years, and exposure threshold=7 minutes. In the present embodiment, the time on ventilator parameter may be measured based on parameters sensed by the ventilator which are indicative of the ventilator's operation and not of spontaneous patient breathing. In another example, the processing instructions may verify that the mechanical ventilator is set to ventilate before evaluating the protocol. In a further example, a parameter indicating that the mechanical ventilator is set to ventilate is included in the protocol. One or more of these examples may be applicable depending on the configuration of the mechanical ventilator. An ARDS protocol may comprise: Time on ventilator≧6 hours, and PaO₂/FiO₂≦200 mm Hg, and Vₜ≧8 mL/kg of ideal body weight, and Pplateau≧30 cm H₂O or Ppeak≧35 cm H₂O, and Age≧16 years, and exposure threshold=7 minutes. Variations of the ALI and ARDS protocols provided above may include more or fewer expressions. In a variation thereof, the tidal volume expression comprises Vₜ≧4 mL/kg of ideal body weight. In a further variation thereof, the tidal volume expression comprises Vₜ≧6 mL/kg of ideal body weight.

FIG. 5 shows view 500 illustrating tab 260, which has been selected. Tab 260 includes an expressions table showing a plurality of expressions of a selected protocol 501 and a plurality of columns 502, 504, 506, 508, 510 and 512 corresponding to, respectively, a parameter computed as a function of other parameters, the parameters of expressions, the condition operators of the expressions, the thresholds of the expressions, Boolean operators logically connecting the expressions and the actual values of the parameters. In the example illustrated in view 500, the exposure parameter 520 and VILI risk parameter 530 are computed based on several expressions. The status of the exposure parameter is a parameter in one of the VILI risk expressions. The foregoing illustration shows how to nest parameters in expressions to define complex protocols. Of course, functions can also be hard-coded to simplify the process of defining protocols. Hard-coded functions may be suitable to automatically transform variables acquired by monitoring devices (e.g. a first data), based on transformation models which may be well known or newly developed, into parameters commonly used by healthcare providers. For example, different sensing techniques may be used to determine first data which may be transformed to the arterial oxygenation parameter. Hard-coded functions may be provided for each sensing technique or monitoring device so that regardless of the technique or device used, a protocol comprising the arterial oxygenation parameter may be used without having to reconfigure it. The different monitoring devices may be associated with transformation functions in a database and system 100 may be configured to automatically select the proper transformation function upon detection of a particular monitoring device. Of course, if the monitoring device senses the arterial oxygenation parameter directly, no transformation is needed and the determination of the arterial oxygenation parameter only requires acquiring the first data.

FIG. 6 shows view 600 illustrating tab 660, which has been selected. Tab 660 includes an expressions table 601 showing a plurality of expressions of a selected lung injury risk monitoring protocol and a plurality of columns 502, 504, 506, 508, 510 and 512 corresponding to, respectively, parameters of expressions included in the protocol. As shown, the parameters include Time on ventilator, PaO₂/FiO₂, Vₜ, Pplateau, Ppeak, Age and exposure time. The protocol includes an exposure portion and an exposure time expression. Lung injury exposure is indicated when the exposure portion of the protocol (including all the expressions except the exposure time expression) is satisfied. In the present embodiment, the processing instructions may be configured to track exposure time when the exposure portion of the protocol is satisfied. In the present example, the exposure portion of the protocol includes an OR operator. Therefore, the exposure portion of the protocol may be satisfied even if not all the expressions are satisfied.

PaO₂ is an exemplary arterial oxygenation parameter. PaO₂ may be obtained by testing arterial blood gases from a blood sample of the patient to assess how well the lungs are oxygenating. Blood samples may be obtained automatically and periodically with an intra-arterial cannula or indwelling arterial catheter. PaO₂ may then be recorded through an indwelling polarograph PO₂ monitoring device. Other arterial oxygenation parameters may also be used to assess how well the lungs are oxygenating. In one example, PO₂ is measured transcutaneously. The measurement may be normalized and correlated to PaO₂ obtained from an arterial blood gas test to calibrate the measured values. In this manner, PO₂ can be measured periodically and automatically to determine PaO₂ even though PaO₂ is directly measured only infrequently. For example, FIG. 4 shows PaO₂ on panel 270 as a manual parameter and also on panel 280 as a monitored device parameter. The PaO₂ value on panel 270 may be obtained infrequently while the value on panel 280 is periodically obtained or computed. By periodically it is meant that samples are obtained at a predetermined rate that corresponds to the protocol being monitored. The predetermined rate may have a period that is a fraction of a second, a second, a few seconds, a minute or a few minutes, in order to provide effective monitoring given the exposure threshold of the protocol. The period may also be determined in relation to the time required by the monitoring devices to measure the actual parameter values. In one example, the period is one minute. In another example, the period is configurable. The period may be configured for each monitoring device and mechanical ventilator such that some devices are monitored more frequently than others.

In another embodiment according to the disclosure, PaO₂ is estimated based on an oxygen saturation value derived from signals obtained with a pulse oximeter and a oxyhemoglobin dissociation curve. Values of an exemplary oxyhemoglobin dissociation curve are shown in the following table. Linear integration between the given points may be performed to estimate values not shown on the table. Furthermore, a curve may be fitted to the given points to extrapolate values below the lower points.

OXYHEMOGLOBIN DISSOCIATION CURVE

| SaO₂ | PaO₂ |
|---|---|
| 50% | 26.6 mmHg |
| 70% | 40.0 mmHg |
| 90% | 60.0 mmHg |
| 95% | 80.0 mmHg |
| 99% | 100.0 mmHg |

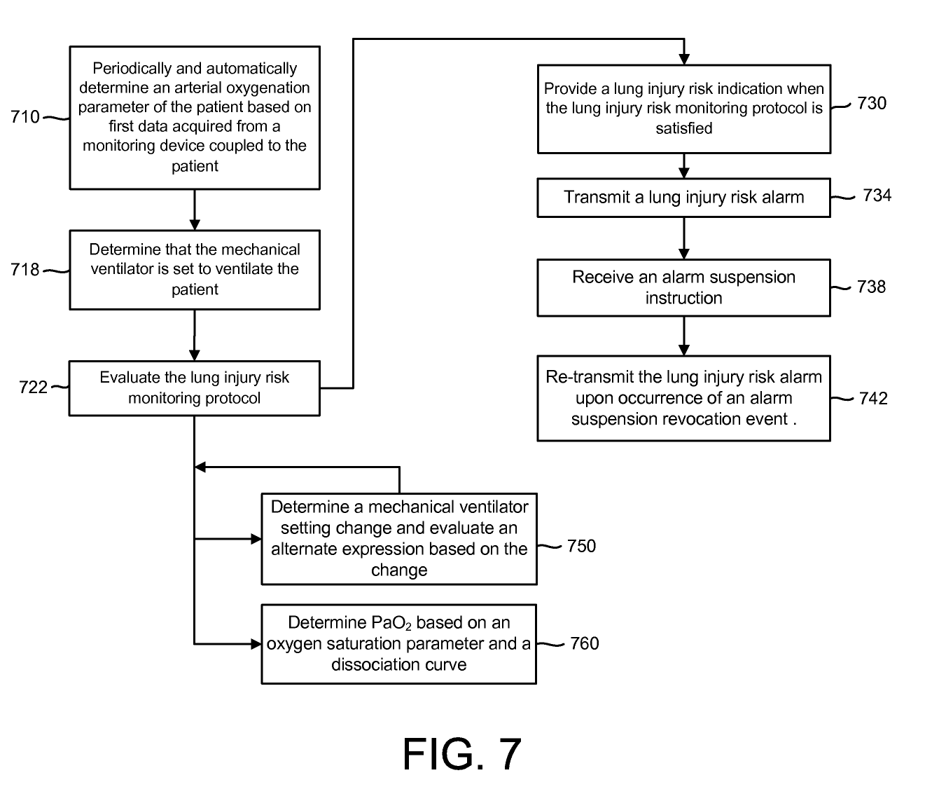

An embodiment of a method according to the disclosure for automatically monitoring lung-protective ventilation of a patient will now be described with reference to FIG. 7. The method may be performed, at least partially, by a processor executing processing instructions embedded in a computer readable storage medium.

As indicated previously, potentially multiple protocols may be defined (e.g., a well known protocol found in research literature, or a doctor's or hospital's own variant protocol). Each protocol is represented as one or more patient parameter relationship expressions. Also, a protocol may comprise a composite expression comprised of multiple parameters and operators. The protocols may be user configured (e.g., users may add, modify, or delete expressions and protocols as desired—for example to differentiate adult ICU patients having been briefly under anesthesia from a pediatric case from a long-term mechanically ventilated patient). A user interface may be operable to define expressions and protocols, select protocols and modify the protocols. A nurse or other healthcare provider may associate the patient with the ventilator. A patient may be automatically associated with a lung injury monitoring protocol, which may be a standard or a predefined protocol when the patient is coupled to the ventilator. If a predefined protocol is modified after it is associated with the patient, the modifications may be stored in the patient configuration file. Also, the modifications may be stored in a new protocol. In one embodiment, a patient is monitored as soon as the data for an associated protocol is received by the system. The system may support evaluation of multiple patients. The healthcare provider may switch views to see the data corresponding to a selected patient, for example by selecting a different patient from a drop-down list. In the meantime, automatic monitoring continues for the other patients.

At 710, the processing instructions cause the processor to periodically and automatically determine an arterial oxygenation parameter of the patient based on first data acquired from a monitoring device coupled to the patient. First data may be acquired at periodic time intervals by the monitoring devices or a data acquisition system and then acquired from the data acquisition system by the lung-protective ventilation monitoring system. The first data may also be acquired by the lung-protective ventilation monitoring system directly from the monitoring devices. The first data may comprise the arterial oxygenation parameter or data operable to determine the arterial oxygenation parameter. In one example, the arterial oxygenation comprises PaO₂ and is provided by the monitoring device. In another example, the monitoring device provides signals operable to determine an oxygen saturation value, and arterial oxygenation is determined by transforming the oxygen saturation value based on a known dissociation curve.

At 718, the processing instructions cause the processor to determine that the mechanical ventilator is set to ventilate the patient. In one variation, the mechanical ventilator communicates a mode parameter which a healthcare provider may select to a ventilation mode or a spontaneous breathing mode. In one example, the processing instructions include instructions to continue processing the method only if the mechanical ventilator is set to ventilate the patient. In another example, the determination that a mechanical ventilator is set to ventilate the patient comprises evaluating parameters sensed by the ventilator indicative of actual ventilation of the patient.

At 722, the processing instructions cause the processor to evaluate a lung injury risk monitoring protocol associated with the patient, the lung injury risk monitoring protocol including the arterial oxygenation parameter. In one variation, at 760, the arterial oxygenation parameter comprises PaO₂, the first data comprises at least one of an oxygen saturation parameter and data operable by the processing instructions to determine the oxygen saturation parameter, and PaO₂ is determined based on the oxygen saturation parameter and a dissociation curve. In another variation, the lung injury risk monitoring protocol comprises a plurality of expressions configured to determine a lung injury risk exposure, and the lung injury risk monitoring protocol is satisfied when an exposure parameter equals or exceeds an exposure threshold.

In a further variation, the protocol is configured to monitor ALI and comprises the expression PaO₂/FiO₂≦300 mm Hg. In another variation, the protocol is configured to monitor ARDS and comprises the expression PaO₂/FiO₂≦200 mm Hg. In a yet further, the protocol includes one or more of the following expressions:
PaO₂/FiO₂≦200 mm Hg,
Tidal volume (Vₜ)≧8 mL/kg of ideal body weight,
Plateau pressure (Pplateau)≧30 cm H₂O or Ppeak≧35 cm H₂O.
Time on ventilator≧6 hours
Age≧16 years
   Age refers to the age of the patient. Time on ventilator refers to the amount of time the patient has been continuously ventilated.

At 730, the processing instructions cause the processor to provide a lung injury risk indication when the lung injury risk monitoring protocol is satisfied. As illustrated in the figures, the lung injury risk indication may be a color or text indication provided by a GUI. The indication may also be non-visual. Non-visual indications include a vibration signal and an aural signal. The indication may be provided in a computer display, a mobile device, the mechanical ventilator, or any other suitable device. The indication may also comprise a visual or non-visual alarm.

At 734, the processing instructions may cause the processor to transmit a lung injury risk alarm.

At 738, the processing instructions may cause the processor to receive an alarm suspension instruction. A healthcare provider receiving the alarm may choose to suspend the alarm via a GUI in the mobile device, a text message, or any other means. The suspension instruction may be transmitted by the mobile device directly or through a facility annunciation system. In one variation, the facility annunciation system may take an action on the alarm without further transmitting the suspension instruction, therefore the processor does not receive the alarm suspension instruction.

At 742, the processing instructions may cause the processor to re-transmit the alarm upon occurrence of an alarm suspension revocation event.

In one variation, at 750, the method further comprises determining a mechanical ventilator setting change and evaluating an alternate expression based on the change.

Additional embodiments of the method described herein may comprise any of the variations and examples of functions executed by processing instructions as described above. The method may also comprise defining a lung injury risk monitoring protocol with a user interface, associating the protocol with a patient, and initiating monitoring with the protocol.

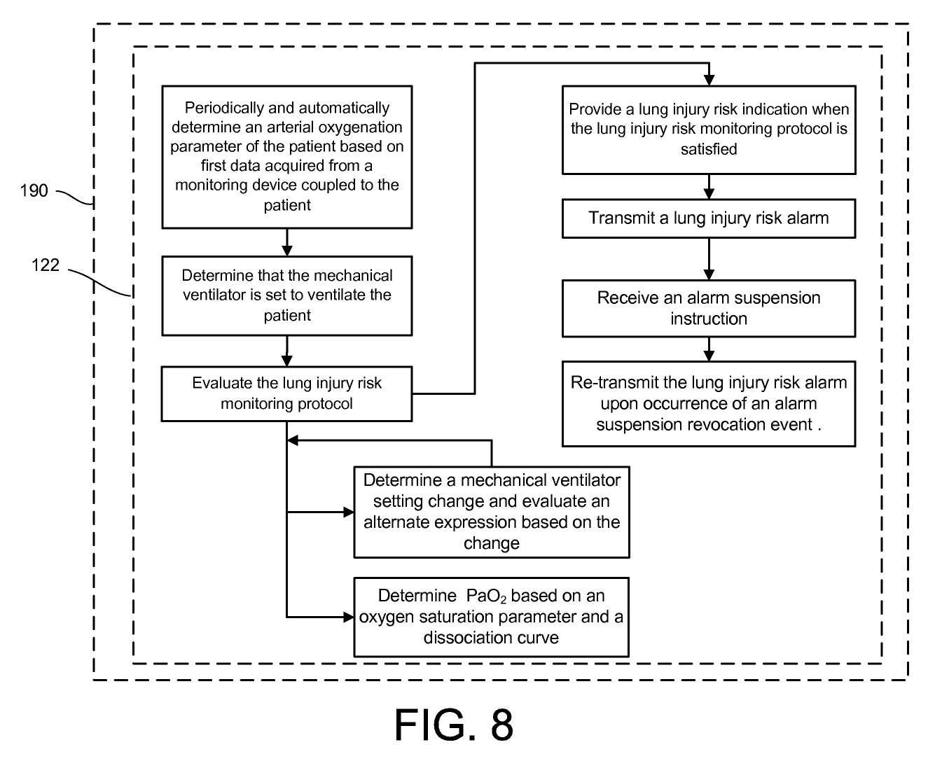

An embodiment according to the disclosure of a computer program product, e.g. computer program product 190, for automatically monitoring a patient's lung injury risk will now be described with reference to FIG. 8. The computer program product comprises a computer readable storage medium having computer readable processing instructions, e.g. processing instructions 122, embedded therein executable by a processor to automatically monitor lung-protective ventilation of a patient. The processing instructions are configured to cause the processor to periodically and automatically determine an arterial oxygenation parameter of the patient based on first data acquired from a monitoring device coupled to the patient; determine that the mechanical ventilator is set to ventilate the patient; evaluate a lung injury risk monitoring protocol associated with the patient, the lung injury risk monitoring protocol including the arterial oxygenation parameter; and provide a lung injury risk indication when the lung injury risk monitoring protocol is satisfied.

Referring again to FIG. 1, system 100 comprises processing instructions 122 and processor 110. As used herein, a software program, algorithm or processing sequence, generally referred to as “processing instructions,” is a self consistent sequence of instructions that can be followed to perform a particular task. Software programs may use data structures for both inputting information and performing the particular task. Data structures greatly facilitate data management. Data structures are not the information content of a memory, rather they represent specific electronic structural elements which impart a physical organization on the information stored in memory. More than mere abstraction, the data structures are specific electrical or magnetic structural elements in memory which simultaneously represent complex data accurately and provide increased efficiency in computer operation. A database is an example of a data structure.

A processing or computing system or device may be a specifically constructed apparatus or may comprise general purpose computers selectively activated or reconfigured by software programs stored therein. The computing device, whether specifically constructed or general purpose, has at least one processing device, or processor, for executing processing instructions and computer readable storage media, or memory, for storing instructions and other information. Many combinations of processing circuitry and information storing equipment are known by those of ordinary skill in these arts. A processor may be a microprocessor, a digital signal processor (DSP), a central processing unit (CPU), or other circuit or equivalent capable of interpreting instructions or performing logical actions on information. A processor encompasses multiple processors integrated in a motherboard and may also include one or more graphics processors and embedded memory. Exemplary processing systems include workstations, personal computers, portable computers, portable wireless devices, mobile devices, and any device including a processor, memory and software. Processing systems also encompass one or more computing devices and include computer networks and distributed computing devices.

As used herein, a computer network, or network, is a system of computing systems or computing devices interconnected in such a manner that messages may be transmitted between them. Typically one or more computers operate as a “server”, a computer with access to large storage devices such as hard disk drives and communication hardware to operate peripheral devices such as printers, routers, or modems. Other computers, termed “clients”, provide a user interface so that users of computer networks can access the network resources, such as shared data files, common peripheral devices, and inter workstation communication. User interfaces comprise software working together with user devices to communicate user commands to the processing system. Exemplary user devices include touch-screens, keypads, mice, voice-recognition logic, imaging systems configured to recognize gestures, and any known or future developed hardware suitable to receive user commands.

A computer readable storage medium comprises any medium configured to store data and includes volatile and non-volatile memory, temporary and cache memory and optical or magnetic disk storage. Exemplary storage media include electronic, magnetic, optical, printed, or media, in any format, used to store information. Computer readable storage medium also comprises a plurality thereof.

Embodiments of the disclosure may be implemented in “object oriented” software. The “object oriented” software is organized into “objects”, each comprising a block of computer instructions describing various procedures to be performed in response to “messages” sent to the object or “events” which occur with the object. Such operations include, for example, the manipulation of variables, the activation of an object by an external event, and the transmission of one or more messages to other objects. Messages are sent and received between objects having certain functions and knowledge to carry out processes. Messages are generated in response to user instructions, for example, by a user activating an icon with a mouse pointer or touch-screen to generate an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access.

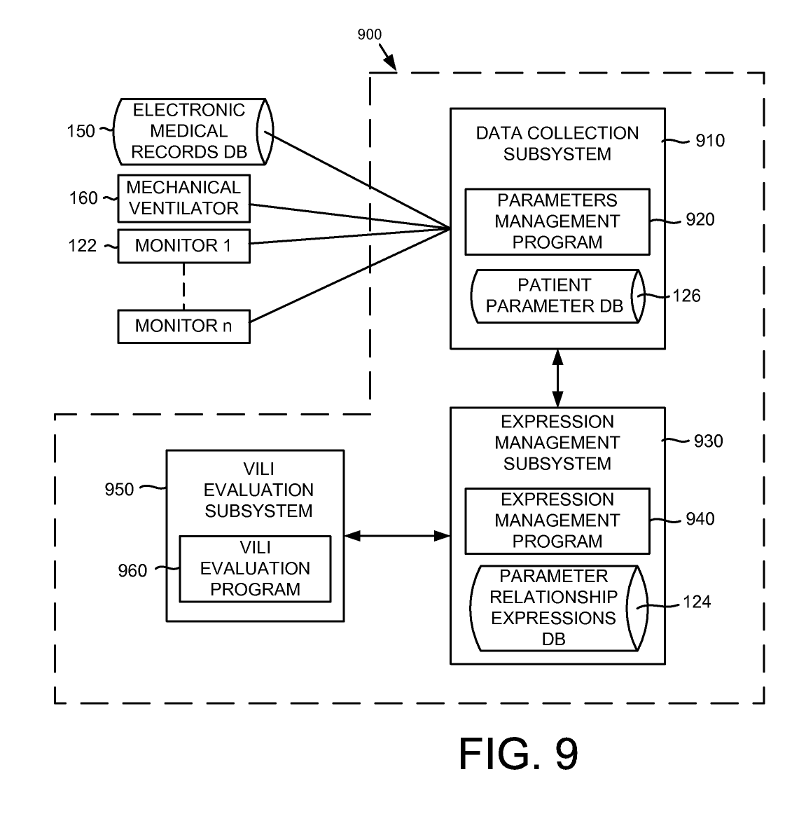

Referring now to FIG. 9, a block diagram of another embodiment of a lung-protection ventilation monitoring system according to the disclosure is presented. In the present embodiment, the system is denoted by numeral 900. As in system 100, system 900 may receive patient parameters from a plurality of data sources including a medical records database, patient monitoring devices, and user interfaces. As shown, system 900 comprises a data collection subsystem 910, an expression management subsystem 930 and a VILI evaluation subsystem 950. Each subsystem comprises a processor configured the execute processing instructions embedded in a computer readable storage medium accessible by the processor. The subsystems are networked and configured so that multiple VILI evaluation subsystem 950 may communicate with one (or more) expression management subsystem 930. Data collection subsystem 910 may be integral with a hospital data collection system. In another embodiment, one or more of subsystems 910, 930 and 950 are combined in a network server.

Data collection subsystem 910 includes a parameters management program 920 and patient parameter database 126. Parameters management program 920 causes a processor to acquire patient parameters from the data sources and to store the patient parameters in patient parameter database 126. Data collection subsystem 910 may also store monitoring device settings information and/or functions and drivers corresponding to monitoring devices. Patient parameters may be normalized prior to being stored in patient parameter database 126.

Expression management subsystem 930 includes an expression management program 940 and parameter relationship expressions database 124. Lung injury risk monitoring protocols may be stored in expression management subsystem 930. Expression management program 940 may cause a processor to acquire patient parameters from data collection subsystem 910 and to evaluate the lung injury risk monitoring protocols. In one example, expression management subsystem 930 may evaluate the protocols upon request from the VILI evaluation subsystem 950. In another example, expression management subsystem 930 may evaluate the protocols periodically. In one example, expression management subsystem 930 communicates the results of the evaluation of each expression and/or protocol to VILI evaluation subsystem 950.

VILI evaluation subsystem 950 comprises a VILI evaluation program 960. VILI evaluation program 960 causes a processor to present a user interface with which a healthcare provider may configure lung injury risk monitoring protocols, as described above, VILI evaluation subsystem 950 may verify that the mechanical ventilator is set to a ventilate the patient and, if so, request expression management subsystem 930 to evaluate the expressions. VILI evaluation subsystem 950 monitors the success or failure of the expressions and the corresponding protocol. Of course, as described above, the mode of the mechanical ventilator may be determinable from a parameter obtained from the mechanical ventilator by expression management subsystem 930 if the parameter is included in an expression, in which case VILI evaluation subsystem 950 may not have to periodically verify that the mechanical ventilator is set to ventilate the patient. In one variation, the lung injury risk monitoring protocols may be stored in a storage medium in VILI evaluation subsystem 950.

In a still further variation of the present embodiment, a selection tool is presented (not shown) to enable a clinician to select a patient parameter or expression. The system then presents a graphical representation comprising historical values of the patient parameter or the expression.

While this invention has been described as having an exemplary design, the present invention may be further modified within the spirit and scope of this disclosure. This application is intended to cover any variations, uses, or adaptations of the invention using its general principles. Further, this application is intended to cover such departures from the present disclosure as come within known or customary practice in the art to which this invention pertains.

What is claimed is:

1. A system to automatically monitor lung-protective ventilation of a patient, the system comprising:
a processor;
a computer readable storage medium; and
processing instructions embedded in the computer readable storage medium and executable by the processor to
periodically and automatically determine an arterial oxygenation parameter of the patient based on first data acquired from a monitoring device coupled to the patient;
determine that the mechanical ventilator is set to ventilate the patient;
evaluate a lung injury risk monitoring protocol associated with the patient, the lung injury risk monitoring protocol including the arterial oxygenation parameter; and
provide a lung injury risk indication when the lung injury risk monitoring protocol is satisfied.

2. A system as in claim 1, wherein the lung injury risk monitoring protocol comprises a plurality of expressions configured to determine a lung injury risk exposure, and the lung injury risk monitoring protocol is satisfied when an exposure parameter equals or exceeds an exposure threshold.

3. A system as in claim 1, wherein the arterial oxygenation parameter comprises partial pressure of arterial oxygen (PaO₂), the first data comprises at least one of an oxygen saturation parameter and data operable by the processing instructions to determine the oxygen saturation parameter, and PaO₂ is determined based on the oxygen saturation parameter and a dissociation curve.

4. A system as in claim 1, wherein the lung injury risk monitoring protocol includes an inspired oxygen fraction (FiO₂), is configured to detect an acute lung injury and comprises the expressions:
PaO₂/FiO₂≦300 mm Hg;
Tidal volume (Vₜ)≧8 mL/kg of ideal body weight; and
Plateau pressure (Pplateau)≧30 cm H₂O or Peak airway pressure (Ppeak)≧35 cm H₂O.

5. A system as in claim 4, wherein the lung injury risk monitoring protocol further comprises the expressions: time on ventilator≧6 hours, and Age≧16 years.

6. A system as in claim 1, wherein the lung injury risk monitoring protocol is configured to detect an acute respiratory distress syndrome and comprises the expressions:
PaO₂/FiO₂≦300 mm Hg;
Tidal volume (Vₜ)≧8 mL/kg of ideal body weight; and
Plateau pressure (Pplateau)≧30 cm H₂O or Peak airway pressure (Ppeak)≧35 cm H₂O.

7. A system as in claim 1, wherein the processing instructions are further configured to determine a change in mechanical ventilator setting and to evaluate an alternate expression based on the change.

8. A method implemented to automatically monitor lung-protective ventilation of a patient, the method comprising:
by a processor, executing processing instructions embedded in a computer readable storage medium, the processing instructions configured to:
periodically and automatically determine an arterial oxygenation parameter of the patient based on first data acquired from a monitoring device coupled to the patient;
determine that the mechanical ventilator is set to ventilate the patient;
evaluate a lung injury risk monitoring protocol associated with the patient, the lung injury risk monitoring protocol including the arterial oxygenation parameter; and
provide a lung injury risk indication when the lung injury risk monitoring protocol is satisfied.

9. A method as in claim 8, wherein the lung injury risk monitoring protocol comprises a plurality of expressions configured to determine a lung injury risk exposure, and the lung injury risk monitoring protocol is satisfied when an exposure parameter equals or exceeds an exposure threshold.

10. A method as in claim 8, wherein the arterial oxygenation parameter comprises PaO₂, the first data comprises at least one of an oxygen saturation parameter and data operable by the processing instructions to determine the oxygen saturation parameter, and PaO₂ is determined based on the oxygen saturation parameter and a dissociation curve.

11. A method as in claim 8, wherein the lung injury risk monitoring protocol is configured to detect an acute lung injury and comprises the expressions:
PaO₂/FiO₂≦300 mm Hg;
Vₜ≧8 mL/kg of ideal body weight; and
Pplateau≧30 cm H₂O or Ppeak≧35 cm H₂O.

12. A method as in claim 8, wherein the lung injury risk monitoring protocol is configured to detect an acute respiratory distress syndrome and comprises the expressions:
PaO₂/FiO₂≦200 mm Hg;
Vₜ≧8 mL/kg of ideal body weight; and
Pplateau≧30 cm H₂O or Ppeak≧35 cm H₂O.

13. A method as in claim 8, further comprising: determining a mechanical ventilator setting change and evaluating an alternate expression based on the change.

14. A computer program product comprising a computer readable storage medium having computer readable processing instructions embedded therein executable by a processor to automatically monitor lung-protective ventilation of a patient, the processing instructions configured to cause the processor to:
periodically and automatically determine an arterial oxygenation parameter of the patient based on first data acquired from a monitoring device coupled to the patient;
determine that the mechanical ventilator is set to ventilate the patient;
evaluate a lung injury risk monitoring protocol associated with the patient, the lung injury risk monitoring protocol including the arterial oxygenation parameter; and
provide a lung injury risk indication when the lung injury risk monitoring protocol is satisfied.

15. A computer program product as in claim 15, wherein the lung injury risk monitoring protocol comprises a plurality of expressions configured to determine a lung injury risk exposure, and the lung injury risk monitoring protocol is satisfied when an exposure parameter equals or exceeds an exposure threshold.

16. A computer program product as in claim 15, wherein the arterial oxygenation parameter comprises PaO₂, the first data comprises at least one of an oxygen saturation parameter and data operable by the processing instructions to determine the oxygen saturation parameter, and PaO₂ is determined based on the oxygen saturation parameter and a dissociation curve.

17. A computer program product as in claim 15, wherein the lung injury risk monitoring protocol is configured to detect an acute lung injury and comprises the expressions:
PaO₂/FiO₂≦300 mm Hg;
Vₜ≧8 mL/kg of ideal body weight, and
Pplateau≧30 cm H₂O or Ppeak≧35 cm H₂O.

18. A computer program product as in claim 17, wherein the at least one lung injury risk expression further comprises the expressions: time on ventilator≧6 hours, and Age≧16 years.

19. A computer program product as in claim 15, wherein the lung injury risk monitoring protocol is configured to detect an acute respiratory distress syndrome and comprises the expressions:
PaO₂/FiO₂≦200 mm Hg;
Vₜ≧8 mL/kg of ideal body weight; and
Pplateau≧30 cm H₂O or Ppeak≧35 cm H₂O.

20. A computer program product as in claim 15, wherein the processing instructions are further configured to determine a mechanical ventilator setting change and to evaluate an alternate expression based on the change.
